(19) United States
(12) Patent Application Publication
(10) Pub. No.: US 2022/0409257 A1
(43) Pub. Date: Dec. 29, 2022

(54) CONTROLLING ESOPHAGEAL TEMPERATURE DURING CARDIAC ABLATION

(71) Applicant: University of Florida Research Foundation, Inc., Gainesville, FL (US)

(72) Inventors: John N. Catanzaro, Jacksonville, FL (US); Michele Dill, Gainesville, FL (US); Christopher Batich, Gainesville, FL (US)

(21) Appl. No.: 17/286,015

(22) PCT Filed: Aug. 28, 2019
(86) PCT No.: PCT/US2019/048512

§ 371 Date: Apr. 16, 2021

Related U.S. Application Data
(60) Provisional application No. 62/746,739, filed on Oct. 17, 2018.

(51) Int. Cl. A61B 18/04; A61F 7/12

(52) U.S. Cl. CPC A61B 18/04; A61F 7/12; A61B 2018/00351

(57) ABSTRACT

A flexible catheter is inserted into the esophagus to cool or warm the esophagus, particularly during certain procedures which can tend to change the temperature in the area of the esophagus. The catheter is inserted through the mouth and throat to a position, for example, proximate the heart, but within the esophagus. One or more balloons are inflated to block areas of the esophagus, while a gel is injected into the esophagus where it is immobilized by the one or more balloons. A coolant is pumped through a coolant tube affixed to the catheter, where it exchanges heat with the conductive gel.

### CROSS-REFERENCE TO RELATED APPLICATION

This application claims priority to and the benefit of Provisional Application No. 62/746,739, entitled “CONTROLLING ESOPHAGEAL TEMPERATURE DURING CARDIAC ABLATION,” and filed Oct. 17, 2018, which is incorporated by reference in its entirety.

### TECHNICAL FIELD

The disclosure relates to a system and method for controlling esophageal temperature during cardiac ablation, and in particular, to changing the temperature in an interior of the esophagus.

### BACKGROUND

Ablation of tissues surrounding the pulmonary veins is carried out to disrupt an electrical signal transmitted from the veins into the left atrium, giving rise to atrial fibrillation. One technique for creating this ablation is the Convergent Procedure, which uses radio frequency energy to generate heat which is applied to heart tissue to produce ablation and interrupt the signal.

Radiofrequency ablation, specifically left atrial endocardial ablation or pulmonary vein isolation in patients with symptomatic paroxysmal or persistent atrial fibrillation uses radiofrequency energy applied to the left atrium at the ostium of the pulmonary veins and sometimes on the posterior wall. An atrial esophageal fistula is a known and debilitating (if not fatal) complication resulting in fistula formation between the atrium and esophagus with entry of air into the left atrium. This may lead to cerebrovascular attack and or myocardial infarction. In addition to standard pulmonary vein isolation, the Convergent Procedure is generally performed in patients with symptomatic persistent atrial fibrillation. An initial part of the procedure utilizes a radio frequency (RF) probe or coil which is placed transdiaphragmatically on an exterior surface of the heart on the posterior wall of the epicardium, in an effort to ablate the epicardial posterior wall. The device utilizes RF energy emitted from a generator which is grounded to the patient. A coil apparatus is introduced telescopically onto the epicardium which then uses a vacuum suction while applying the RF energy. The impedance is measured while RF is applied in an effort to confirm that the application of energy is complete, and that sufficient energy has been transmitted to the epicardium in order to cause ablation.

To complete a desired ablation pattern near the blood vessels, ablation is additionally performed inside the heart using electrophysiology. A device is threaded through the femoral artery into the heart, and RF energy is again used to complete portions of the ablation pattern which could not be completed outside the heart.

Cryothermal energy has been used inside the heart on the endocardium to ablate the ostium of pulmonary veins, including for example by use of the ARTIC FRONT device of Medtronic, Inc. The device occludes the ostium with a round balloon-like structure which is inserted into the ostium to make contact with body tissue, and which is then filled with a coolant to cause freezing of tissue at the ostium.

Laser ablation has also been used to isolate the pulmonary veins in symptomatic paroxysmal atrial fibrillation via an endoscopic balloon introduced transseptally into the left atrium. The probe is placed into the pulmonary vein and the balloon is deployed giving the operator visualization of the pulmonary vein before applying laser application. Laser application can increase left atrial temperature and predispose the esophagus to collateral damage via thermal injury.

All of the above modalities have a latent effect of energy, that is, when stopping radiofrequency, or laser, the temperature measured in the esophagus continues to rise to a plateau before nadir. Cryothermal may have the same effect but in an opposite direction “freeze”.

### SUMMARY

Aspects of the present disclosure are related to apparatuses and methods for cooling or warming an interior area of the esophagus during a therapeutic procedure.

In one aspect, among others, a device for cooling or warming an interior area of the esophagus during a therapeutic procedure comprises an elongated, flexible catheter having a proximal end and distal end; a proximal balloon affixed to an exterior surface of the catheter relative to the proximal end of the catheter, the proximal balloon being configured and sized to block a proximal portion of the esophagus when inflated; a distal balloon affixed to an exterior surface of the distal end of the catheter, the distal balloon being configured and sized to block a second portion of the esophagus when inflated; at least one balloon inflation lumen extending through the catheter having at least one inflation inlet in communication with the interior of the proximal balloon and the distal balloon; and a gel injection lumen extending through the catheter having a gel inlet in fluid communication with a gel outlet positioned at the distal end of the catheter and between the proximal balloon and the distal balloon.

In various aspects, the device comprises a coolant tube having a coolant inlet in communication with a coolant outlet. The coolant tube is affixed to the surface of the catheter, extending from the proximal end of the catheter to the distal end of the catheter, proximal to the distal balloon, and then back to the proximal end of the catheter. In various aspects, the coolant tube is coiled around the outer surface of the catheter.

In various aspects, the coolant tube is filled with at least one of a carbon or a metal. In various aspects, the coolant tube has an outer diameter of about 1.7 millimeters. In various aspects, the coolant inlet is attached to a pump configured to pump a heated or cooled fluid through the coolant tube. In various aspects, more temperatures sensors are connected to the tube and configured to output temperature information pertaining to the interior area of the esophagus. In various aspects, a steerable element is inserted into an interior of the catheter and is configured to be bent when positioned inside the body and in the interior of the catheter to thereby cause a change in an orientation of the catheter within the body.

In another aspect, among others, a kit comprises the device and a polymeric material for producing a gel. In another aspect, among other, a kit comprises the device and a gel. In various aspects, the gel comprises water and a polyalkylene glycol. In various aspects, the polyalkylene glycol comprises polyethylene glycol, polypropylene glycol, monomethoxy polyethylene glycol, a poloxamer, or any combination thereof. In various aspects, the polyalkylene glycol has a molecular weight of about 600 Da to about 6,000 Da. In various aspects, the polyalkylene glycol is from about 0.1 wt % to 5 wt % of the gel. In various aspects, the gel has a dielectric constant of less than 20. In various aspects, the gel comprises a thermally conductive gel.

In another aspect, among others, a method for cooling or warming an interior area of the esophagus during a therapeutic procedure comprises inserting the device in any one of claims 1-8 into the esophagus; inflating a proximal balloon and a distal balloon of the device to block a proximal section and a distal section of the esophagus; and injecting a gel into the gel injection lumen of the device in order to deposit the gel into the esophagus, the gel being deposited into the esophagus in an area below the proximal balloon and above the distal balloon. In various aspects, the gel comprises water and a polyalkylene glycol. In various aspects, the polyalkylene glycol comprises polyethylene glycol, polypropylene glycol, monomethoxy polyethylene glycol, a poloxamer, or any combination thereof. In various aspects, the polyalkylene glycol has a molecular weight of about 600 Da to about 6,000 Da. In various aspects, the polyalkylene glycol is from about 0.1 wt % to 5 wt % of the gel. In various aspects, the gel has a dielectric constant of less than 20. In various aspects, the gel comprises a thermally conductive gel.

In another aspect, among others, a device for in vitro testing atrial ablation devices, comprises a flexible tube having a lumen configured and sized to mimic an esophagus; a hydrogel positioned on the surface of the flexible tube, wherein the hydrogel is sized and configured to mimic a left atrial wall; a heat source configured to heat the hydrogel to ablative temperatures; and at least one of a first temperature probe positioned in the lumen of the flexible tube and a second temperature probe positioned between the flexible tube and the hydrogel. In various aspects, the heat source is configured to produce a temperature of at least 150° C. In various aspects, the flexible tube has an inner diameter of about 2 cm and a thickness of about 5 mm. In various aspects, the hydrogel has a thickness of at least 5 mm. In various aspects, a third temperature probe is positioned in the hydrogel at least 3 mm from the flexible tube. In various aspects, the device is suspended in a saline bath at 37° C.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### DESCRIPTION OF DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 depicts an example of a device for cooling the esophagus during an atrial ablation according to various embodiments of the present disclosure.

FIG. 2 depicts another example of a device for cooling the esophagus during an atrial ablation according to various embodiments of the present disclosure.

FIG. 3 depicts another example of a device for cooling the esophagus during an atrial ablation according to various embodiments of the present disclosure.

FIG. 4 depicts another example of a device for cooling the esophagus during an atrial ablation according to various embodiments of the present disclosure.

FIG. 5 depicts a device of the disclosure for mimicking the spatial relationship and thermal conductivity of the left atrium and esophagus according to various embodiments of the present disclosure.

FIG. 6 depicts a wireless communication device, some or all of which can be used in carrying out the disclosure according to various embodiments of the disclosure.

FIG. 7 is an example of a graph showing change in temperature at the esophageal wall of an in vitro testing platform mimicking esophageal temperature during atrial ablation according to various embodiments of the present disclosure.

FIG. 8 is an example of a graph showing change in temperature at the esophageal wall as in FIG. 7 using a device of the disclosure for cooling the esophagus during an atrial ablation according to various embodiments of the present disclosure.

FIG. 9 is an example image of a device for cooling the esophagus during an atrial ablation according to various embodiments of the present disclosure.

FIG. 10 is an example image of a device for cooling the esophagus during an atrial ablation according to various embodiments of the present disclosure.

### DETAILED DESCRIPTION

As required, detailed embodiments are disclosed herein; however, it is to be understood that the disclosed embodiments are merely examples and that the systems and methods described below can be embodied in various forms. Therefore, specific structural and functional details disclosed herein are not to be interpreted as limiting, but merely as a basis for the claims and as a representative basis for teaching one skilled in the art to variously employ the present subject matter in virtually any appropriately detailed structure and function. Further, the terms and phrases used herein are not intended to be limiting, but rather, to provide an understandable description of the concepts.

The terms “a” or “an”, as used herein, are defined as one or more than one. The term plurality, as used herein, is defined as two or more than two. The term another, as used herein, is defined as at least a second or more. The terms “including” and “having,” as used herein, are defined as comprising (i.e., open language). The term “coupled,” as used herein, is defined as “connected,” although not necessarily directly, and not necessarily mechanically.

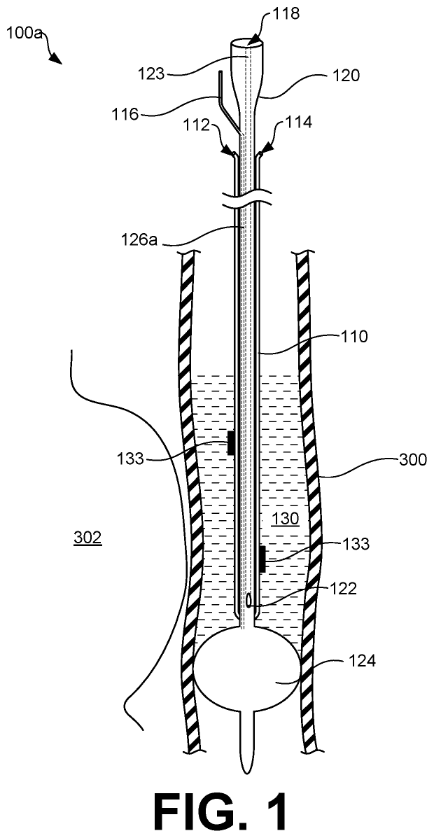

In accordance with the disclosure, open or closed loop irrigation of cooling or warming liquid is applied to an interior surface of the esophagus that is proximate to the heart during a procedure which is applying heat or cold to the heart, and particularly in the region of the left atrium 302 (FIG. 1) which is most proximate the esophagus. Such procedures can include ablation of the heart using heat, such as during radio frequency or laser ablation, or ablation of the heart using cooling, such as during cryoablation. A temperature-control device of the disclosure is used to apply a cooling substance to an interior surface of the esophagus during heat ablation, and a warming substance during cold ablation. The applied substance acts as a medium to counterbalance a resultant and undesired change in temperature of the esophagus, which can otherwise damage the esophagus during the therapeutic treatment of the heart. Such damage can include the formation of ulcers or an esophageal fistula, for example. The disclosure provides medical devices that improve the safety of left atrial ablation by reducing the incidence and severity of esophageal injury, by providing for targeted temperature control.

According to various embodiments of the present disclosure, the temperature-control device can also help the electrophysiologist or cardiothoracic surgeon gain information on which types of lesion orientation from the catheter (sliding, parallel or perpendicular can afford a transmurality lesion while preserving the integrity of the esophagus). The disclosure provides closed, semi-closed, and open loop irrigation devices and methods according to various embodiments.

Turning now to FIGS. 1-4, shown are examples of temperature-control devices 100 (e.g., 100a, 100b, 100c, 100d) for cooling the esophagus during an atrial ablation according to various embodiments of the present disclosure. The temperature-control device includes a catheter 120 comprising a flexible elongated body that extends from a proximal end to a distal end. The catheter 120 is configured and sized to be passable from outside of the body to the interior area of the esophagus 300.

According to various embodiments, the temperature control device 100 can comprise a coolant tube 110 having an inlet 112 and an outlet 114 that is disposed around the catheter 120. The coolant tube 110 can be made of any biocompatible material that is water impermeable but with adequate thermal conductivity to transfer heat from the system. For example, the coolant 110 can be made of silicone, PVC, natural rubber, styrene butadiene rubber, polyisobutene, polyethylene vinylacetate, ethylene-propylene di-monomer (EPDM), nylons, PET, fluoro-containing co-polymers such as perfluoroethylene-propylene, polypropylene, polyacrylonitrile, polyvinyl alcohol, and/or other type of material as can be appreciated. According to various embodiments, the coolant tube 110 can be filled with carbon, graphene, and/or metal particles to increase thermal conductivity. In some embodiments, the coolant tube 110 does not have to be very flexible, and a thin-walled metal could be used as well if it could be bent without kinking.

The inner and outer diameter of the coolant tube 110 can be selected based on the material used to provide sufficient flow and thermal conductivity. For example, in some embodiments, the coolant tube 110 can have an outer diameter of about 0.5 to 8.0 mm. As an example, the coolant tube 110 can have an outer diameter of 1.7 mm and an inner diameter of 0.76 mm. In some embodiments, the coolant inlet is attached to a pump (not shown) configured to pump a heated or cooled fluid through the coolant tube 110. The fluid preferably has a high specific heat. The fluid can comprise water, saline, capable of being sacredly ingested such as an emulsion of fat in water that does not damage the material of the device, and/or any other type of fluid as can be appreciated.

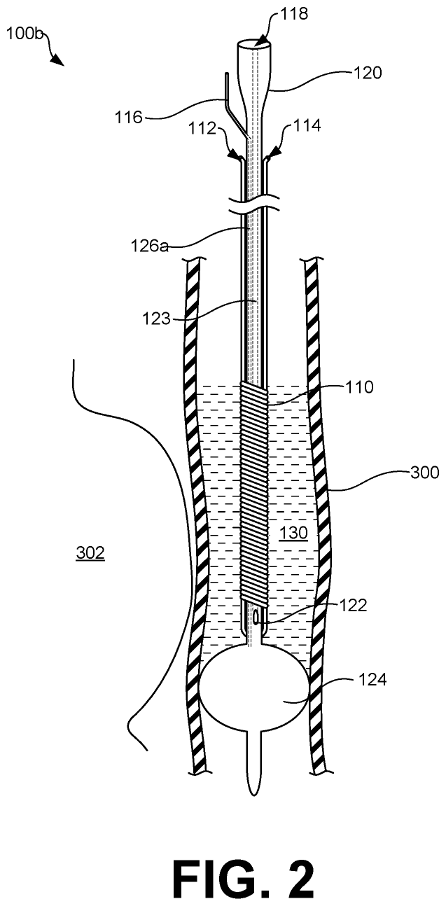

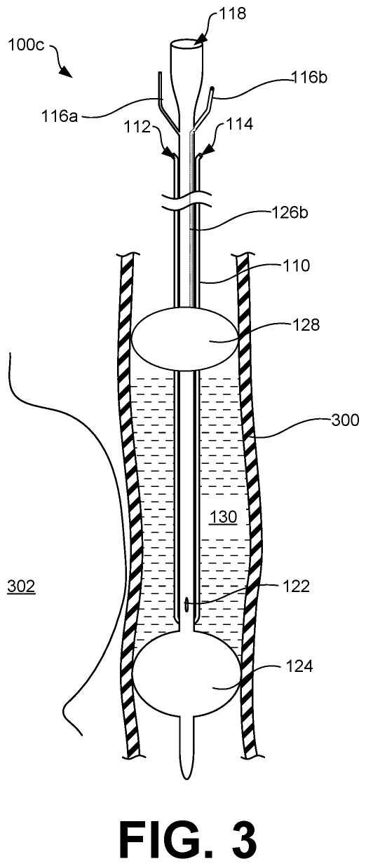

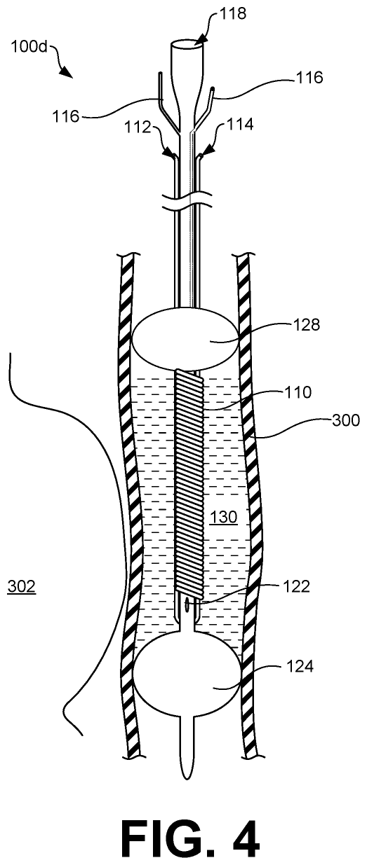

FIGS. 1 and 3 illustrate examples of the coolant tube 110 extending longitudinally along the length of the catheter 120 with a single bend at the distal end before extending longitudinally back along the catheter and out of the body. In other examples, as shown in FIGS. 2 and 4, the coolant tube 110 can be coiled with one or more loops around the outer surface of the catheter 120. Although the coolant tube 110 in FIGS. 2 and 4 illustrate a coil with multiple loops surrounding the catheter 120, the coolant tube 110 can comprise a coil around the catheter 120 having one or more loops. For example, the coolant tube 110 can extend towards a distal end of the catheter 120 and loop at least one time around the catheter 120 before returning towards the proximal end of the catheter 120. It should be noted that while the coolant tube 110 is described as a coil with one or more loops around the catheter 120 or a tube that runs lengthwise along the catheter 120, the coolant tube 110 can be formed in any other shape or pattern for optimal surface area as can be appreciated.

The catheter 120 includes a gel inlet 118 at its proximal end and a gel port 122 fluidly connected to the gel inlet 118 via a gel lumen 123 extending through the catheter 120. The gel port 122 is positioned about the catheter 120 and configured to release a gel 130 injected in the gel inlet 118 through the gel lumen 123 and into the esophagus 300 to serve as a medium for convective heat exchange.

According to various embodiments, as shown in FIGS. 1-4, the catheter 120 also includes a distal inflatable balloon 124 at the distal end of the catheter 120. The distal inflatable balloon 124 is fluidly connected to an inflation inlet 116 (e.g., 116a, 116b) at the proximal end of the catheter 120 through a balloon inflation lumen 126 (e.g., 126a, 126b) that extends through the catheter 120. The inflation inlet 116 is configured and coupled to the distal balloon 124 such that injection of an inflation fluid into the inflation inlet 116 inflates the distal balloon 124 to a size sufficient to block the esophagus 300 and trap the gel 130 above the distal balloon 124 to prevent the gel 130 from entering the stomach. The distal balloon 124 can be in a deflated state during insertion and removal of the temperature-controlling device 100 into an esophagus or other suitable area.

As shown in FIGS. 3 and 4, temperature-controlling device 100 can comprise a proximal balloon 128 positioned at a proximal portion of the catheter 120 above the gel port 122 of the catheter 120. The proximal balloon 128 is fluidly connected to an inflation inlet 116 such that injection of an inflation fluid into the inflation inlet 116 inflates the proximal balloon 128 to a size sufficient to block the esophagus 300 and trap the gel 130 below the proximal balloon 128 to prevent the gel 130 from moving into the lungs. The proximal balloon 128 can be in a deflated state during insertion and removal of the temperature-controlling device 100 into an esophagus 300 or other suitable area.

According to various embodiments, the proximal balloon 128 is disposed around an outer surface of the catheter 120. In some embodiments, the proximal balloon 128 surrounds the catheter 120 and at least a portion of the coolant tube 110 disposed along the catheter 120. Although shown separately in FIGS. 3 and 4, in some embodiments, the inflation inlet 116 that is fluidly coupled to the proximal balloon 128 is the same inflation inlet 116 that is fluidly coupled to the distal balloon 124 such that inflation fluid travels through the same balloon inflation lumen 126 of the catheter 120. In other embodiments, the inflation inlet 116 that is fluidly coupled to the proximal balloon 128 is separate from the inflation inlet 116 that is fluidly coupled to the distal balloon 124. For example, the catheter 120 may comprise a second balloon inflation lumen 126b that extends through the catheter to an entry point of the proximal balloon 128. In other embodiments, the inflation inlet 116 is coupled to a tube (not shown) having a balloon inflation lumen 125 that is coupled to the proximal balloon 128 and separate from the catheter 120.

According to various embodiments, the inflation fluid can comprise air, saline, and/or other types of inflation fluids capable of being sacredly ingested such as an emulsion of fat in water that does not damage the material of the device as can be appreciated. In addition, although the proximal balloon 128 is described as an inflatable balloon, in some embodiments, the proximal balloon 128 can comprise an expandable sponge and/or other material that can be used to trap the gel 130 below the proximal balloon 128, sponge, and/or other suitable component.

For example, although FIGS. 3 and 4 illustrate a proximal balloon 128, the proximal balloon 128 can comprise a sponge. According to various embodiments, an expandable sponge for example, can be at least partially dried so that it has a smaller than maximum dimension, enabling device 100 to be more easily inserted into the esophagus 300 and positioned at a site of therapy, for example near to the heart. It may be desired to retain some moisture within sponge, for example, to ensure that the surface thereof is soft and resilient, to protect body tissue. When inserted, the sponge can expand to contact inner surfaces of esophagus 300 and trap the gel 130 below the sponge. In some embodiments, cooled or heated fluid can then be circulated through tubes 110 as described elsewhere herein, to transfer or remove heat to or from the sponge or proximal balloon 128. In some embodiment, expandable biocompatible material within a sponge can then transfer the heat or cold to the inner surface of the esophagus 300, providing the intended therapeutic benefit.

According to various embodiments, the device 100 can comprise a temperature sensor 133. As shown in FIG. 1, a temperature sensor 133 can also be positioned at one or more locations along catheter 120. This sensor 133 can transmit temperature data corresponding to an adjacent area along the esophagus 300. A plurality of sensors 133 can provide temperature information for a plurality of areas along esophagus 300, whereby signal processing equipment connected to sensors 133 can identify particular areas of the esophagus which are experiencing or anticipated to experience an undesired temperature change. Sensors 133 can transmit this data via wires, or by a wireless communication, such as WIFI, BLUETOOTH or other nearfield protocol, and any other wireless protocol. To avoid inadvertent heating of sensor by radiofrequency associated with RF ablation, it can be advantageous to avoid using metal in sensor. This may be achieved using a fiber optic sensor, for example. Other non-metallic or metallic temperature sensor technologies may be used for sensor.

In an embodiment, an electronic processor 802 receives temperature information from sensors 133, and reports elevated temperatures. In some cases, the electronic processor 802 controls rate of flow through the coolant tube 110.

In some embodiments, the device 100 further includes a steerable element (not shown) inserted into an interior of the catheter 120, the steerable element configured to be bent when positioned inside the body and in the interior of the catheter 120 to thereby cause a change in an orientation of the catheter 120 within the body.

As discussed above, in certain aspects, one or more gels 130 can be used with the devices 100 described herein that can be readily cooled or heated as needed. The gels 130 are formulated such that they can be injected into the esophagus via the devices 100 as described herein. The gels 130 are composed of water and a non-toxic polymeric material suitable for administration to a subject.

The selection of the polymeric material can vary. In one aspect, the polymeric material is a polyalkylene glycol. “Polyalkylene glycol” as used herein refers to a condensation polymer of ethylene oxide or propylene oxide and water. Polyalkylene glycols are typically colorless liquids with high molecular weights and are soluble in water as well as some organic solvents. In one aspect, the polyalkylene glycol is polyethylene glycol and/or polypropylene glycol. In another aspect, the polyalkylene glycol is monomethoxy polyethylene glycol. In one aspect, the polyalkylene glycol is Miralax® (polyethylene glycol having an average molecular weight of 3,350 manufactured by Bayer) or Carbowax™ (polyethylene glycol having an average molecular weight of 600 to 6,000 manufactured by Dow Chemical).

In one aspect, the polyalkylene glycol is a poloxamer. Poloxamers are nonionic triblock copolymers composed of a central hydrophobic chain of polyoxypropylene (e.g., (poly(propylene oxide)) flanked by two hydrophilic chains of polyoxyethylene (e.g., poly(ethylene oxide)). In one aspect, poloxamer has the formula

HO(C₂H₄O)_{b}(C₃H₆O)ₐ(C₂H₄O)_{b}OH

wherein a is from 10 to 100, 20 to 80, 25 to 70, or 25 to 70, or from 50 to 70; b is from 5 to 250, 10 to 225, 20 to 200, 50 to 200, 100 to 200, or 150 to 200. In another aspect, the poloxamer has a molecular weight from 2,000 Da to 15,000 Da, 3,000 Da to 14,000 Da, or 4,000 Da to 12,000 Da. Poloxamers useful herein are sold under the tradename Pluronic® manufactured by BASF.

When a polyalkylene glycol is used to produce the gel 130, the gel 130 has a low dielectric constant. In one aspect, the dielectric constant of the gel 130 less than 20, or is about 10, about 11, about 12, about 13, about 14, about 15, about 16, about 17, about 18, about 19, or less than about 20, where any value can be a lower or upper endpoint of a range (e.g., about 11 to about 18, about 13 to about 16, etc.) as measured with a TR-1A Ratio Arm Transformer Bridge from Ando Electric Co. In another aspect, the polyalkylene glycol has a molecular weight of about 600 Da to about 6,000 Da, or about 600 Da, about 750 Da, about 1,000 Da, about 1,500 Da, about 2,500 Da, about 3,000 Da, about 3,500 Da, about 4,000 Da, about 4,500 Da, about 5,000 Da, about 5,500 Da, about 6,000 Da, where any value can be a lower or upper endpoint of a range (e.g., about 600 Da to about 2,000 Da, about 3,000 Da to about 5,000 Da, etc.). However, it should be noted molecular weights greater than 6,000 Da for the polyalkylene glycol can be used as can be appreciated. According to various embodiments, the pure polyethylene polymers have dielectric constants of about 20, and their solutions will be some average with that of water.

In other aspects, the polymeric material used to produce the gel 130 is a thermally conductive gel. Examples of polymeric materials used to produce thermally conductive gels include, but are not limited to, alginate, xanthan gum, or gelatin. In another aspect, the thermally conductive gel is any non-toxic thickener for water or saline. In one aspect, the polymeric material used to produce the thermally conductive gel comprises chitosan or acacia. In another aspect, the polymeric material used to produce the thermally conductive gel comprises arrowroot, corn starch, katakuri starch, potato starch, sago, tapioca, or their starch derivatives. In another aspect, the thermally conductive gel comprises microbial and vegetable gums used as food thickeners including, but not limited to, alginin, guar gum, locust bean gum, and xanthan gum. In other aspects, the thermally conductive gel comprises inorganic thickeners such as sodium pyrophosphate.

The gel 130 can be prepared by admixing the polymeric material in water with one or more optional components as needed. The admixing of the polymeric material with water can be conducted at room temperature or at elevated temperatures depending upon the selection and amount of polymeric material used. In one aspect, the gel has a viscosity high enough to allow the inflated balloon to be immobilize it in the esophagus, but low enough to allow it to be injected and aspirated. The amount of polymeric material used to produce the gel can be modified in order to fine-tune the viscosity of the gel. In one aspect, the polymeric material is from about 0.1 wt % to 5 wt % of the gel, or is about 0.1 wt %, about 0.5 wt %, about 1.0 wt %, about 1.5 wt %, about 2.0 wt %, about 2.5 wt %, about 3.0 wt %, about 3.5 wt %, about 4.0 wt %, about 4.5 wt %, or about 5.0 wt %, where any value can be a lower or upper endpoint of a range (e.g., about 0.1 wt % to about 4.0 wt %, about 0.5 wt % to about 2.0 wt %, etc.).

In certain aspects, the gel 130 can be produced when it is time to use the device described herein. In one aspect, a kit comprising the device 100 described herein can include the components to produce the gel 130. For example, the polymeric material can be provided as a dry material in the kit with instructions for admixing the polymeric material with a certain volume of water. In other aspects, the kit can include the device 100 with the gel 130 already prepared for use. The kits described herein can also include one or more syringes for injecting the gel into the devices described herein.

Devices 100 of the disclosure can be used in the various manners described herein, and can additionally be advantageously used in outflow tract tachycardia or right ventricle ablations in the epicardium, particularly where the endocardium is thin. Additionally, according to various embodiments, a temperature-controlling device 100 can be used to buffer the convective heat introduced from an ablation catheter, enabling in certain cases transmittal of full thickness lesions with a lower chance of perforation or collateral damage to adjacent epicardial arteries than in cases where devices 100 are not used.

Devices 100 of the disclosure can have any size which can be effectively inserted into the esophagus of a given patient, which varies widely according to human anatomy. An example non-limiting range of diameter includes about 4 mm to 20 mm, and lengths of about 250 to 500 centimeters (cm). Smaller, wider, longer, or shorter sizes can be used depending upon the patient size, whether or not it is desired for the cooling/warming area of the device to contact the esophageal wall, and a length extending outside of the body that is convenient. Appropriate biocompatible materials can be used, as understood within the art, although the avoidance of metal is advantageous to avoid undesired retransmission of RF energy within the esophagus 300.

Flexible components are advantageously made with a biocompatible polymer with sufficient flexibility, durability, and lubricity, as would be understood within the art. Examples can include Poly(ethylene) (PE) (HDPE, UHMWPE); Poly(propylene) (PP); Poly(tetrafluroethylene) (PTFE) (Teflon), expanded-PTFE; Ethylene-co-vinylacetate (EVA); Poly(dimethylsiloxane) (PDMS); Poly(ether-urethanes) (PU); Poly(ethylene terphthalate) (PET); and Poly(sulphone) (PS), although other materials, including polymeric, synthetic, and natural, can be used.

According to various embodiments, the catheter 120 can be advanced into the esophagus 300 of a subject such that the distal balloon 124 is advanced passed the atrium. The distal balloon 124 and the proximal balloon 128 can then be inflated to the point that it blocks areas of the esophagus 300. A gel 130 can then be injected into the esophagus 300, where it is immobilized by the distal balloon 124 and the proximal balloon 128. A coolant can be pumped through the coolant tube 110 to transfer heat away from the gel 130. At this point, the subject is ready for atrial ablation. Once that is completed, the balloons 124, 128 can be deflated and the catheter 120 retracted. In some embodiments, the device 100 further comprises a means for aspirating the gel 130 out of the esophagus 300 prior to deflating the balloons 124, 128. In some cases, this is accomplished through the gel port 122. In other embodiments, the device 100 comprises an aspiration lumen (not shown) that extends through the catheter 120 to an aspiration inlet (not shown) at the distal end of the catheter 120, proximal to the distal balloon 124.

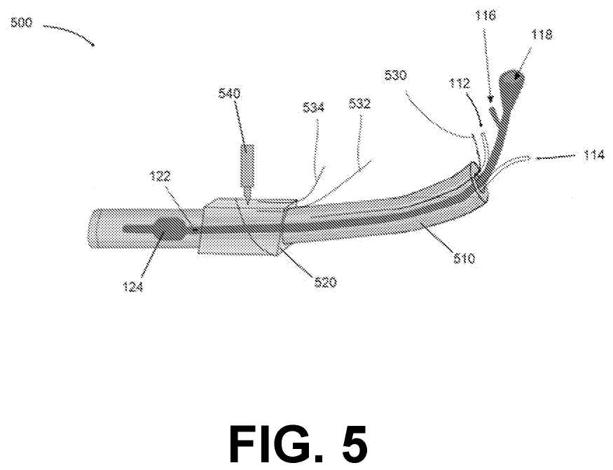

Turning now to FIG. 5, depicted is a testing device 500 being used to test the ability of a device 100 of the disclosure for cooling or warming an interior area of the esophagus 300 during a therapeutic procedure. The testing device 500 includes a flexible tube 510 having a lumen configured and sized to mimic an esophagus 300. The testing device 500 further includes a hydrogel 520 positioned on the surface of the flexible tube, wherein the hydrogel 520 is sized and configured to mimic a left atrial wall. The testing device 500 can further includes a heat or cooling source 540 configured to heat or cool the hydrogel to ablative temperatures. The testing device 500 can further include one of a first temperature probe 530 positioned in the lumen of the flexible tube and a second temperature probe 532 positioned between the flexible tube and the hydrogel. Other temperature probes and sensors can be included. For example, the device 500 can further include a third temperature probe 534 positioned in the hydrogel 520 at least 3 mm from the flexible tube, e.g. at or near the surface of the hydrogel 520 where heat will be applied.

The heat source can be any device configured to produce an ablative effect. In some cases, the heat source involve radiofrequency energy, which is typically used at powers of up to 35 Watts and is delivered through an irrigated ablation catheter. In some cases, heat source involves a laser. In some cases, the heat source is cryogenic and actually removes heat. In these embodiments, the disclosed device transfers heat to the esophagus instead of away from the esophagus.

The flexible tube 510 is sized to mimic a human esophagus. Therefore, in some embodiments, the flexible tube has an inner diameter of about 2 cm and a thickness of about 5 mm.

The hydrogel 520 is sized and configured to mimic a left atrial wall. Therefore, in some embodiments, the hydrogel has a thickness of about 5 mm. The hydrogel 520 can be made of any material with a thermal conductivity approximating that of the atrial wall. For example, in some embodiments, the hydrogel is 5% agar. Other suitable materials include synthetic polymers such a polyvinyl alcohol or polyhydroxyethylmethacryate (pHEMA).

The device 500 is also configured so that it can be suspended in a 37° C. saline bath. In some cases, the flexible tube 510 is affixed to a stand and/or placed in a container to provide structural support.

In some embodiments, the device 500 further includes one or more temperatures sensors connected to the tube 510 and configured to output temperature information pertaining to the interior.

In some embodiments, a steerable element is inserted into the catheter lumen, the steerable element configured to be bent when positioned inside the body and in the interior of the catheter, to thereby cause a change in an orientation of the catheter within the body.

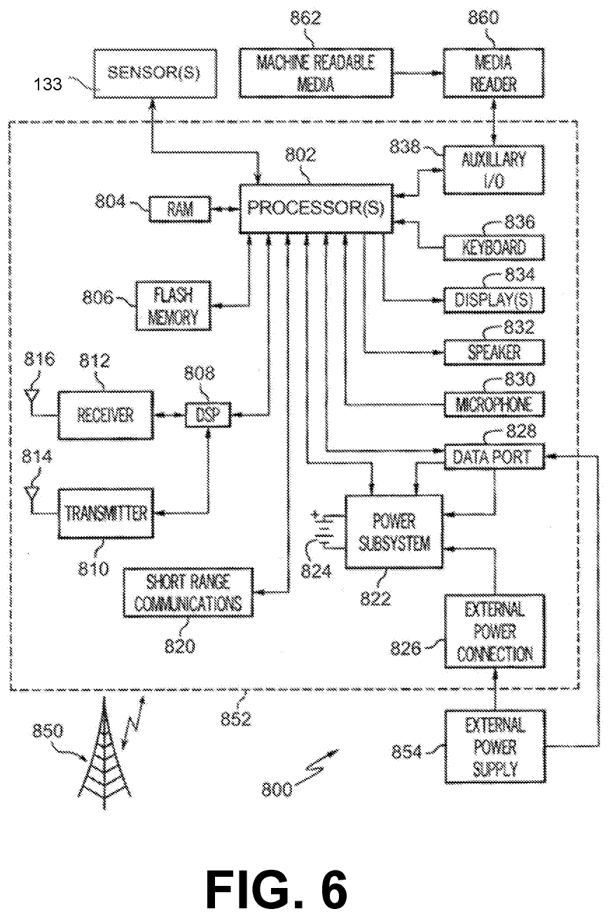

FIG. 6 is a block diagram of an electronic device and associated components 800, which can be used in carrying out the disclosure. In this example, an electronic device 852 is a wireless two-way communication device with voice and data communication capabilities. Such electronic devices communicate with a wireless voice or data network 850 using a suitable wireless communications protocol. Wireless voice communications are performed using either an analog or digital wireless communication channel. Data communications allow the electronic device 852 to communicate with other computer systems via the Internet. Examples of electronic devices that are able to incorporate the above described systems and methods include, for example, a data messaging device, a two-way pager, a cellular telephone with data messaging capabilities, a wireless Internet appliance or a data communication device that may or may not include telephony capabilities. Electronic device 800 can be used, for example, to gather electronic data from sensors 133 by wired or wireless means, to display such data or otherwise communicate such data to medical practitioners, and to control flow of cool or warm fluid through device 100.

The illustrated electronic device 852 is an example electronic device that includes two-way wireless communications functions. Such electronic devices incorporate communication subsystem elements such as a wireless transmitter 810, a wireless receiver 812, and associated components such as one or more antenna elements 814 and 816. A digital signal processor (DSP) 808 performs processing to extract data from received wireless signals and to generate signals to be transmitted. The particular design of the communication subsystem is dependent upon the communication network and associated wireless communications protocols with which the device is intended to operate.

The electronic device 852 includes a microprocessor 802 that controls the overall operation of the electronic device 852. The microprocessor 802 interacts with the above described communications subsystem elements and also interacts with other device subsystems such as flash memory 806, random access memory (RAM) 804, auxiliary input/output (I/O) device 838, data port 828, display 834, keyboard 836, speaker 832, microphone 830, a short-range communications subsystem 820, a power subsystem 822, and any other device subsystems.

A battery 824 is connected to a power subsystem 822 to provide power to the circuits of the electronic device 852. The power subsystem 822 includes power distribution circuitry for providing power to the electronic device 852 and also contains battery charging circuitry to manage recharging the battery 824. The power subsystem 822 includes a battery monitoring circuit that is operable to provide a status of one or more battery status indicators, such as remaining capacity, temperature, voltage, electrical current consumption, and the like, to various components of the electronic device 852.

The data port 828 of one example is a receptacle connector 104 or a connector that to which an electrical and optical data communications circuit connector (not shown) engages and mates, as described above. The data port 828 is able to support data communications between the electronic device 852 and other devices through various modes of data communications, such as high speed data transfers over an optical communications circuits or over electrical data communications circuits such as a USB connection incorporated into the data port 828 of some examples. Data port 828 is able to support communications with, for example, an external computer or other device.

Data communication through data port 828 enables a user to set preferences through the external device or through a software application and extends the capabilities of the device by enabling information or software exchange through direct connections between the electronic device 852 and external data sources rather than via a wireless data communication network. In addition to data communication, the data port 828 provides power to the power subsystem 822 to charge the battery 824 or to supply power to the electronic circuits, such as microprocessor 802, of the electronic device 852.

Operating system software used by the microprocessor 802 is stored in flash memory 806. Further examples are able to use a battery backed-up RAM or other non-volatile storage data elements to store operating systems, other executable programs, or both. The operating system software, device application software, or parts thereof, are able to be temporarily loaded into volatile data storage such as RAM 804. Data received via wireless communication signals or through wired communications are also able to be stored to RAM 804.

The microprocessor 802, in addition to its operating system functions, is able to execute software applications on the electronic device 852. A predetermined set of applications that control basic device operations, including at least data and voice communication applications, is able to be installed on the electronic device 852 during manufacture. Examples of applications that are able to be loaded onto the device may be a personal information manager (PIM) application having the ability to organize and manage data items relating to the device user, such as, but not limited to, e-mail, calendar events, voice mails, appointments, and task items.

Further applications may also be loaded onto the electronic device 852 through, for example, the wireless network 850, an auxiliary I/O device 838, data port 828, short-range communications subsystem 820, or any combination of these interfaces. Such applications are then able to be installed by a user in the RAM 804 or a non-volatile store for execution by the microprocessor 802.

In a data communication mode, a received signal such as a text message or web page download is processed by the communication subsystem, including wireless receiver 812 and wireless transmitter 810, and communicated data is provided the microprocessor 802, which is able to further process the received data for output to the display 834, or alternatively, to an auxiliary I/O device 838 or the Data port 828. A user of the electronic device 852 may also compose data items, such as e-mail messages, using the keyboard 836, which is able to include a complete alphanumeric keyboard or a telephone-type keypad, in conjunction with the display 834 and possibly an auxiliary I/O device 838. Such composed items are then able to be transmitted over a communication network through the communication subsystem.

For voice communications, overall operation of the electronic device 852 is substantially similar, except that received signals are generally provided to a speaker 832 and signals for transmission are generally produced by a microphone 830. Alternative voice or audio I/O subsystems, such as a voice message recording subsystem, may also be implemented on the electronic device 852. Although voice or audio signal output is generally accomplished primarily through the speaker 832, the display 834 may also be used to provide an indication of the identity of a calling party, the duration of a voice call, or other voice call related information, for example.

Depending on conditions or statuses of the electronic device 852, one or more particular functions associated with a subsystem circuit may be disabled, or an entire subsystem circuit may be disabled. For example, if the battery temperature is low, then voice functions may be disabled, but data communications, such as e-mail, may still be enabled over the communication subsystem.

A short-range communications subsystem 820 provides for data communication between the electronic device 852 and different systems or devices, which need not necessarily be similar devices. For example, the short-range communications subsystem 820 includes an infrared device and associated circuits and components or a Radio Frequency based communication module such as one supporting Bluetooth® communications, to provide for communication with similarly-enabled systems and devices, including the data file transfer communications described above.

A media reader 860 is able to be connected to an auxiliary I/O device 838 to allow, for example, loading computer readable program code of a computer program product into the electronic device 852 for storage into flash memory 806. One example of a media reader 860 is an optical drive such as a CD/DVD drive, which may be used to store data to and read data from a computer readable medium or storage product such as computer readable storage media 862. Examples of suitable computer readable storage media include optical storage media such as a CD or DVD, magnetic media, or any other suitable data storage device. Media reader 860 is alternatively able to be connected to the electronic device through the data port 828 or computer readable program code is alternatively able to be provided to the electronic device 852 through the wireless network 850.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Development of in Vitro Testing Platform

An in vitro testing stand for prototype testing was developed with the goal of mimicking the spatial relationship and thermal conductivity of the left atrium 302 and esophagus 300. The esophagus 300 was represented by a flexible polyvinyl alcohol (PVA) foam tube, with an inner diameter of about two (2) centimeters (cm) and a thickness of about five (5) millimeters (mm.) The inside of the PVA tube was coated with a silicone sealant to limit the porosity of the tube. A 5% agar hydrogel with an outer thickness of 5 mm was used as a phantom of the left atrial tissue. Heat was applied using a soldering iron, and the entire system was submerged in a 37° C. 0.7% saline water bath. The agar tissue phantom was submerged 1 mm below the surface of the saline to mimic surface flow of blood while also reducing heat loss at the ablation site. Tests were performed to compare the agar tissue phantom and soldering iron heat application with previously developed in vitro models that utilized ablation catheters to validate the testing platform. The final rendition of the testing platform is shown in FIG. 5.

### Example 2: Development and Testing of Prototypes

A prototype design (FIGS. 1 and 2) was created that utilizes the balloon of a Foley catheter as a mechanism for blocking the flow of a viscous liquid down the esophagus. The Foley catheter was modified by blocking the lowest port and creating a new port above the distal balloon 124. Silicone tubing was attached to the catheter to transport room temperature water through the device, with one inlet and one outlet. During use, the catheter is inserted into the esophagus model and the distal balloon 124 is positioned below the ablation site to avoid pushing the esophagus towards the left atrium 302. The distal balloon 124 is then inflated with 5 milliliters (mL) of air to block the esophagus and secure the position of the device. Once the prototype is in place, the inlet tube for room temperature water is attached to a pump and the outlet tube is placed in a waste beaker. This pump system can be replaced with a peristaltic pump. Following the establishment of fluid flow through the silicone tubing, 8 mL of a viscous liquid (alginate, xanthan gum, etc.) at room temperature is delivered through the large port of the Foley catheter using a syringe. The purpose of the viscous liquid is to serve as the medium for convective heat exchange and to remain above the inflated balloon.

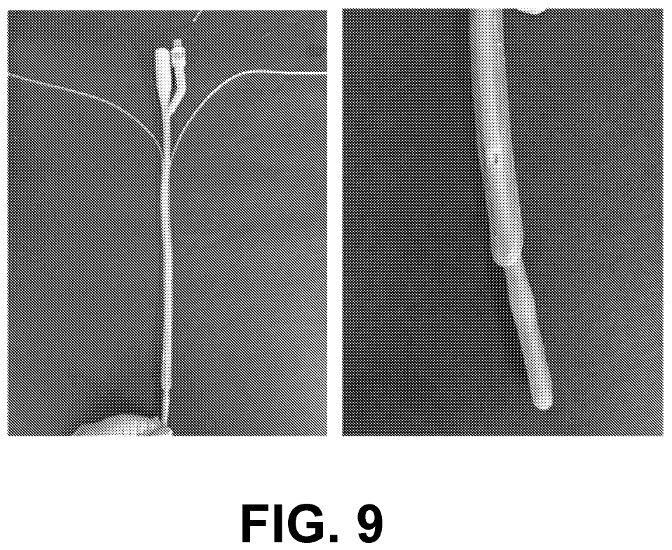

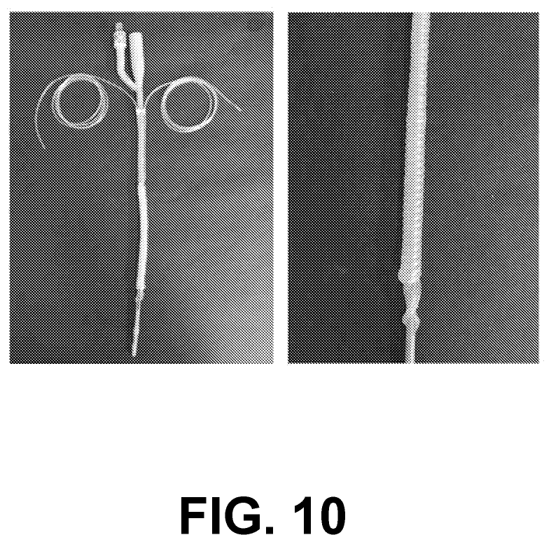

Experiments were performed to determine the proper size and configuration of the silicone tubing for optimum convection prompted by the room temperature water circulation. FIG. 1 shows a one-turn tubing configuration. Experiments were performed to compare changes in temperature 5 mm below the surface of the agar tissue phantom (representative of the esophageal wall) using silicone tubing with a 1.2 mm outer diameter or a 1.7 mm outer diameter in either a one-turn (FIGS. 1, 3, and 9) or coiled (FIGS. 2, 4, and 10) configuration. Based on experimental results, the 1.7 mm outer diameter tubing was selected. The larger tubing size was demonstrated to decrease heating 5 mm below the ablation site when compared to the smaller size tubing. This is thought to be due to the increased flow rate in the 1.7 mm tubing. There was no significant difference observed between the one-turn and coiled conformations following preliminary testing, but the coiled conformation was ultimately selected due to its ability to create a more uniform area of convection within the esophagus.

The 1.7 mm OD coiled silicone tubing prototype has a diameter of 0.85 cm (25.5F) at the point of largest width.

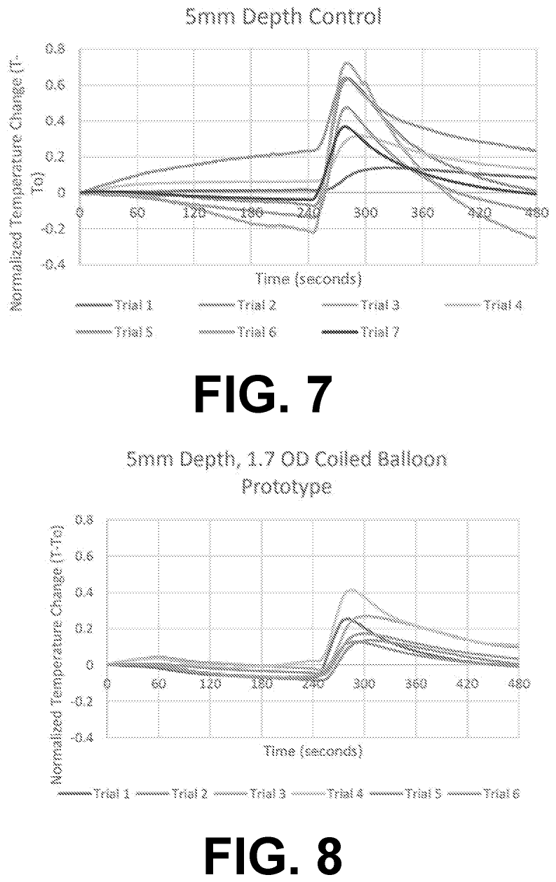

FIGS. 7 and 8 show results comparing the 1.7 mm OD coiled balloon prototype (FIG. 8) to a control (FIG. 7). During these experiments, heat was applied for 30 seconds with a soldering iron at 150° C. at 240 seconds. Temperatures at the site of heat application, 5 mm below the site of heat application, and within the PVA esophagus model were taken over time. The results shown are indicative of the temperature change at the 5 mm depth over time, normalized by the temperature change at the surface. This normalization is performed to account for differences in surface heating due to differences in water height above the site of heat application or differences in heat application using the soldering iron.

The balloon prototype resulted in decreased temperature changes 5 mm below the ablation site, which in this experimental platform is used to indicate the esophageal wall. The data is normalized by the increase in temperature at the ablation site, so the in vitro model asserts that at equal ablation site temperatures, the increase in temperature at the esophageal wall would be lower when the prototype is in use. Specifically, the average maximum temperature at the 5 mm depth for the prototypes were an average of 1.2° C. lower than that of the controls, with average maximum temperatures of 36.8° C. and 38° C. respectively.

One modification of this prototype would be to replace the closed loop convective flow of room temperature water with an open loop flow of the viscous fluid. In this case, the bulk viscous liquid would be replaced over time, potentially increasing the convective heat transfer.

Candidates for the viscous liquid included xanthan gum, alginate, and gelatin between 0.5% and 2% concentrations. Gelatin was ultimately excluded due to a phase change from a gel to a liquid around 27° C. Xanthan gum has been the most widely used in these studies due to ease of preparation (including the experiments in FIG. 6). Xanthan gum can be incorporated into water by stirring alone, while alginate requires heating to allow the polymer to degrade. Experiments are still being performed with 1% and 2% concentrations of xanthan gum and alginate to determine any differences.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Examples of the embodiments of the present disclosure can be described in view of the following clauses:

Clause 1. A device for cooling or warming an interior area of the esophagus during a therapeutic procedure, comprising: an elongated, flexible catheter having a proximal end and distal end; a proximal balloon affixed to an exterior surface of the catheter relative to the proximal end of the catheter, the proximal balloon being configured and sized to block a proximal portion of the esophagus when inflated; a distal balloon affixed to an exterior surface of the distal end of the catheter, the distal balloon being configured and sized to block a second portion of the esophagus when inflated; at least one balloon inflation lumen extending through the catheter having at least one inflation inlet in communication with the interior of the proximal balloon and the distal balloon; and a gel injection lumen extending through the catheter having a gel inlet in fluid communication with a gel outlet positioned at the distal end of the catheter and between the proximal balloon and the distal balloon.

Clause 2. The device of clause 1, further comprising a coolant tube having a coolant inlet in communication with a coolant outlet, wherein the coolant tube is affixed to the surface of the catheter, extending from the proximal end of the catheter to the distal end of the catheter, proximal to the distal balloon, and then back to the proximal end of the catheter.

Clause 3. The device of clause 1 or 2, wherein the coolant tube is coiled around the outer surface of the catheter.

Clause 4. The device of clause 2 or 3, wherein the coolant tube is composed of carbon.

Clause 5. The device of any one of clauses 2 to 4, wherein the coolant tube has an outer diameter of about 1.7 millimeters.

Clause 6. The device of any one of clauses 1 to 6, further comprising one or more temperatures sensors connected to the tube and configured to output temperature information pertaining to the interior area of the esophagus.

Clause 7. The device of any one of clauses 1 to 7, further comprising a steerable element inserted into an interior of the catheter, the steerable element configured to be bent when positioned inside the body and in the interior of the catheter to thereby cause a change in an orientation of the catheter within the body.

Clause 8. The device of any one of clauses 1 to 7, further comprising a steerable element inserted into an interior of the catheter, the steerable element configured to be bent when positioned inside the body and in the interior of the catheter to thereby cause a change in an orientation of the catheter within the body.

Clause 9. A kit comprising the device in any one of clauses 1-8 and a polymeric material for producing a gel.

Clause 10. A kit comprising the device in any one of clauses 1-8 and a gel.

Clause 11. The kit of clauses 9 or 10, wherein the gel comprises water and a polyalkylene glycol.

Clause 12. The kit of clause 11, wherein the polyalkylene glycol comprises polyethylene glycol, polypropylene glycol, monomethoxy polyethylene glycol, a poloxamer, or any combination thereof.

Clause 13. The kit in any one of clauses 10-12, wherein the polyalkylene glycol has a molecular weight of about 600 Da to about 6,000 Da.

Clause 14. The kit in any of clauses 11 to 13, wherein the polyalkylene glycol is from about 0.1 wt % to 5 wt % of the gel.

Clause 15. The kit in any of clauses 9-14, wherein the gel has a dielectric constant of less than 20.

Clause 16. The kit of clause 10, wherein the gel comprises a thermally conductive gel.

Clause 17. A method for cooling or warming an interior area of the esophagus during a therapeutic procedure comprising: inserting the device in any one of claims 1-8 into the esophagus; inflating a proximal balloon and a distal balloon of the device to block a proximal section and a distal section of the esophagus; and injecting a gel into the gel injection lumen of the device in order to deposit the gel into the esophagus, the gel being deposited into the esophagus in an area below the proximal balloon and above the distal balloon.

Clause 18. The method of clause 17, wherein the gel comprises water and a polyalkylene glycol.

Clause 19. The method of clause 18, wherein the polyalkylene glycol comprises polyethylene glycol, polypropylene glycol, monomethoxy polyethylene glycol, a poloxamer, or any combination thereof.

Clause 20. The method in any of clauses 18 or 19, wherein the polyalkylene glycol has a molecular weight of about 600 Da to about 6,000 Da.

Clause 21. The method in any of clauses 18-20, wherein the polyalkylene glycol is from about 0.1 wt % to 5 wt % of the gel.

Clause 22. The method in any of clauses 17-21, wherein the gel has a dielectric constant of less than 20.

Clause 23. The method of clause 17, wherein the gel comprises a thermally conductive gel.

Clause 24. A device for in vitro testing atrial ablation devices, comprising a flexible tube having a lumen configured and sized to mimic an esophagus; a hydrogel positioned on the surface of the flexible tube, wherein the hydrogel is sized and configured to mimic a left atrial wall; a heat source configured to heat the hydrogel to ablative temperatures; and at least one of a first temperature probe positioned in the lumen of the flexible tube and a second temperature probe positioned between the flexible tube and the hydrogel.

Clause 25. The device of clause 24, wherein the heat source is configured to produce a temperature of at least 150° C.

Clause 26. The device of clause 24 or 25, wherein the flexible tube has an inner diameter of about 2 cm and a thickness of about 5 mm.

Clause 27. The device of any one of clauses 24 to 26, wherein the hydrogel has a thickness of at least 5 mm.

Clause 28. The device of any one of clauses 24 to 27, further comprising a third temperature probe positioned in the hydrogel at least 3 mm from the flexible tube.

Clause 29. The device of any one of clauses 24 to 28, suspended in a saline bath at 37° C.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A device for cooling or warming an interior area of the esophagus during a therapeutic procedure, comprising:
an elongated, flexible catheter having a proximal end and distal end;
a proximal balloon affixed to an exterior surface of the catheter relative to the proximal end of the catheter, the proximal balloon being configured and sized to block a proximal portion of the esophagus when inflated;
a distal balloon affixed to an exterior surface of the distal end of the catheter, the distal balloon being configured and sized to block a second portion of the esophagus when inflated;
at least one balloon inflation lumen extending through the catheter having at least one inflation inlet in communication with the interior of the proximal balloon and the distal balloon; and
a gel injection lumen extending through the catheter having a gel inlet in fluid communication with a gel outlet positioned at the distal end of the catheter and between the proximal balloon and the distal balloon.

2. The device of claim 1, further comprising a coolant tube having a coolant inlet in communication with a coolant outlet, wherein the coolant tube is affixed to the surface of the catheter, extending from the proximal end of the catheter to the distal end of the catheter, proximal to the distal balloon, and then back to the proximal end of the catheter.

3. The device of claim 1, wherein the coolant tube is coiled around the outer surface of the catheter.

4. The device of claim 2, wherein the coolant tube is filled with at least one of a carbon or a metal.

5. The device of claim 2, wherein the coolant tube has an outer diameter of about 1.7 millimeters.

6. The device of claim 2, wherein the coolant inlet is attached to a pump configured to pump a heated or cooled fluid through the coolant tube.

7. The device of claim 1, further comprising one or more temperatures sensors connected to the tube and configured to output temperature information pertaining to the interior area of the esophagus.

8. The device of claim 1, further comprising a steerable element inserted into an interior of the catheter, the steerable element configured to be bent when positioned inside the body and in the interior of the catheter to thereby cause a change in an orientation of the catheter within the body.

9. A kit comprising the device in claim 1 and a polymeric material for producing a gel.

10. A kit comprising the device in claim 1 and a gel.

11. The kit of claim 9, wherein the gel comprises water and a polyalkylene glycol.

12. The kit of claim 11, wherein the polyalkylene glycol comprises polyethylene glycol, polypropylene glycol, monomethoxy polyethylene glycol, a poloxamer, or any combination thereof.

13. The kit in claim 10, wherein the polyalkylene glycol has a molecular weight of about 600 Da to about 6,000 Da.

14. The kit in claim 11, wherein the polyalkylene glycol is from about 0.1 wt % to 5 wt % of the gel.

15. The kit in claim 9, wherein the gel has a dielectric constant of less than 20.

16. The kit of claim 10, wherein the gel comprises a thermally conductive gel.

17. A method for cooling or warming an interior area of the esophagus during a therapeutic procedure comprising:
inserting the device in claims 1-8 into the esophagus;
inflating a proximal balloon and a distal balloon of the device to block a proximal section and a distal section of the esophagus; and
injecting a gel into the gel injection lumen of the device in order to deposit the gel into the esophagus, the gel being deposited into the esophagus in an area below the proximal balloon and above the distal balloon.

18. The method of claim 17, wherein the gel comprises water and a polyalkylene glycol.

19. The method of claim 18, wherein the polyalkylene glycol comprises polyethylene glycol, polypropylene glycol, monomethoxy polyethylene glycol, a poloxamer, or any combination thereof.

20. The method in claim 18, wherein the polyalkylene glycol has a molecular weight of about 600 Da to about 6,000 Da.

21. The method in claim 18, wherein the polyalkylene glycol is from about 0.1 wt % to 5 wt % of the gel.

22. The method in claim 17, wherein the gel has a dielectric constant of less than 20.

23. The method of claim 17, wherein the gel comprises a thermally conductive gel.

24. A device for in vitro testing atrial ablation devices, comprising
a flexible tube having a lumen configured and sized to mimic an esophagus;
a hydrogel positioned on the surface of the flexible tube, wherein the hydrogel is sized and configured to mimic a left atrial wall;
a heat source configured to heat the hydrogel to ablative temperatures; and
at least one of a first temperature probe positioned in the lumen of the flexible tube and a second temperature probe positioned between the flexible tube and the hydrogel.

25. The device of claim 24, wherein the heat source is configured to produce a temperature of at least 150° C.

26. The device of claim 24, wherein the flexible tube has an inner diameter of about 2 cm and a thickness of about 5 mm.

27. The device of claim 24, wherein the hydrogel has a thickness of at least 5 mm.

28. The device of claim 24, further comprising a third temperature probe positioned in the hydrogel at least 3 mm from the flexible tube.

29. The device of 24 to 28, suspended in a saline bath at 37° C.
